(19) United States
(12) Patent Application Publication
(10) Pub. No.: US 2004/0254190 A1
(43) Pub. Date: Dec. 16, 2004

(54) 3-Benzhydrylidene-8-aza-bicyclo[3.2.1]octane derivatives with opioid receptor activity

(75) Inventor: Spiros Liras, Stonington, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/825,008

(22) Filed: Apr. 14, 2004

Related U.S. Application Data
(60) Provisional application No. 60/462,886, filed on Apr. 15, 2003.

(51) Int. Cl. A61K 31/52; A61K 31/506; A61K 31/502; A61K 31/501; A61K 31/497

(52) U.S. Cl. 514/248; 514/304; 546/125; 546/126; 544/277; 544/333; 544/405; 544/237; 544/238; 514/252.04; 514/263.22; 514/255.05; 514/256

(57) ABSTRACT

The present invention relates to compounds of the formula I,

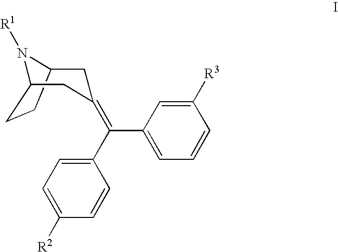

wherein R¹, R², and R³ are defined as in the specification, pharmaceutical compositions containing such compounds, and the use of such compounds to treat neurological and gastrointestinal disorders.

### BACKGROUND OF THE INVENTION

This invention relates to tropane derivatives as opioid drugs. This invention also relates to pharmaceutical compositions comprising such derivatives and the use of such derivatives in the treatment of a variety of neurological and gastrointestinal disorders.

Opioid drugs are typically classified by their binding selectivity with respect to the cellular and differentiated tissue receptors to which a specific drug species binds as a ligand. These receptors include mu (μ), delta (δ) and kappa (κ) receptors.

At least three subtypes of opioid receptors (mu, delta and kappa) are described and documented in the scientific literature. All three receptors are present in the central and peripheral nervous systems of many species including man. Activation of delta receptors produces antinociception in rodents and can induce analgesia in man, in addition to influencing motility of the gastrointestinal tract. (See Burks, T. F. (1995) in “The Pharmacology of Opioid Peptides”, edited by Tseng, L. F., Harwood Academic Publishers).

The well known narcotic opiates such as morphine and its analogs are selective for the opioid mu receptor. Mu receptors mediate analgesia, respiratory depression, and inhibition of gastrointestinal transit. Kappa receptors mediate analgesia and sedation.

The discovery of the opioid delta receptor followed the isolation and characterization of endogenous enkephalin peptides, which are ligands for the delta receptor. Research in the past decade has produced significant information about the delta receptor, but a clear picture of its function has not yet emerged. Delta receptors mediate analgesia, but do not appear to inhibit intestinal transit in the manner characteristic of mu receptors.

U.S. Pat. No. 4,816,586, which issued on Mar. 28, 1989 to P. S. Portoghese, refers to various delta opioid receptor antagonists. These compounds are described as possessing a unique opioid receptor antagonist profile, and include compounds that are highly selective for the delta opioid receptor.

U.S. Pat. No. 4,518,711, which issued May 21, 1985 to V. J. Hruby et al., refers to cyclic, conformationally constrained analogs of enkephalins. These compounds include both agonists and antagonists for the delta receptor and are said to induce pharmacological and therapeutic effects, such as analgesia in the case of agonist species of such compounds. The antagonist species of the disclosed compounds are suggested as useful in the treatment of schizophrenia, Alzheimer's disease, and respiratory and cardiovascular functions. The foregoing patents are incorporated herein by reference in their entirety.

WO 00/14066 discloses certain biarylpiperidine derivatives as selective delta opioid ligands. The foregoing application is owned in common with the present application and is incorporated by reference herein in its entirety.

### SUMMARY OF THE INVENTION

This invention relates to compounds of the formula

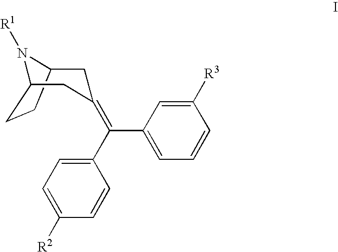

wherein R¹ is hydrogen, (C₁-C₈)alkoxy-(C₁-C₈)alkyl-, wherein the total number of carbon atoms is eight or less, aryl, aryl-(C₁-C₈)alkyl-, heteroaryl, heteroaryl-(C₁-C₈)alkyl-, heterocyclic, heterocyclic-(C₁-C₈)alkyl, (C₃-C₇)cycloalkyl-, or (C₃-C₇)cycloalkyl-(C₁-C₈)alkyl, wherein said aryl and the aryl moiety of said aryl-(C₁-C₈)alkyl- are independently selected from phenyl and napthyl, and wherein said heteroaryl and the heteroaryl moiety of said heteroaryl-(C₁-C₈)alkyl- are independently selected from pyrazinyl, benzofuranyl, quinolyl, isoquinolyl, benzothienyl, isobenzofuryl, pyrazolyl, indolyl, isoindolyl, benzimidazolyl, purinyl, carbazolyl, 1,2,5-thiadiazolyl, quinazolinyl, pyridazinyl, pyrazinyl, cinnolinyl, phthalazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, oxazolyl, oxadiazolyl, isoxazoyl, thiazolyl, isothiazolyl, furanyl, pyrazolyl, pyrrolyl, tetrazolyl, triazolyl, thienyl, imidazolyl, pyridinyl, and pyrimidinyl; and wherein said heterocyclic and the heterocyclic moiety of said heterocyclic-(C₁-C₈)alkyl- are selected from saturated or unsaturated nonaromatic monocyclic or bicyclic ring systems, wherein said monocyclic ring systems contain from four to seven ring carbon atoms, from one to three of which may optionally be replaced with O, N or S, and wherein said bicyclic ring systems contain from seven to twelve ring carbon atoms, from one to four of which may optionally be replaced with O, N or S; and wherein any of the aryl, heteroaryl or heterocyclic moieties of R¹ may optionally be substituted with from one to three substitutuents, preferably with one or two substutituents, independently selected from halo, (C₁-C₆)alkyl optionally substituted with from zero to seven (preferably with from zero to four) fluorine atoms, phenyl, benzyl, hydroxy, acetyl, amino, cyano, nitro, (C₁-C₆)alkoxy, (C₁-C₆)alkylamino and [(C₁-C₆)alkyl]₂amino, and wherein any of alkyl moieties in R¹ (e.g., the alkyl moieties of alkyl, alkoxy or alkylamino groups) may optionally be substituted with from zero to seven (preferably with from zero to four) fluorine atoms;

R² is hydrogen, aryl, heteroaryl, heterocyclic, —SO₂R⁴, —COR⁴, —CONR⁵R⁶, —COOR⁴, or —C(OH)R⁵R⁶ wherein each of R⁴, R⁵ and R⁶ is independently defined as R¹ is defined above, or R⁵ and R⁶, together with the carbon or nitrogen to which they are both attached, form a three to seven membered saturated ring containing from zero to three heterocarbons independently selected from O, N and S, and wherein said aryl, heteroaryl, and heterocyclic are defined as such terms are defined above in the definition of R¹, and wherein any of the aryl, heteroaryl and heterocyclic moieties of R² may optionally be substituted with from one to three substitutuents, preferably with one or two substutituents, independently selected from halo, (C₁-C₆)alkyl optionally substituted with from zero to seven (preferably with from zero to four) fluorine atoms, phenyl, benzyl, hydroxy, acetyl, amino, cyano, nitro, (C₁-C₆)alkoxy optionally substituted with from zero to seven (preferably with from zero to four) fluorine atoms, (C₁-C₆)alkylamino and [(C₁-C₆)alkyl]₂amino;

R³ is hydroxy, —NHSO₂R⁷, —C(OH)R⁷R⁸, fluorine or —CONHR⁷, wherein R⁷ and R⁸ are the same or different and are selected from hydrogen, (C₁-C₄)alkyl, (C₁-C₄)alkoxy and (C₁-C₄)alkoxy-(C₁-C₄)alkyl having a total of 4 or less carbon atoms, and wherein any of the alkyl moieties of R⁷ and R⁸ may optionally be substituted with from zero to seven (preferably with from zero to four) fluorine atoms; and

and the pharmaceutically acceptable salts of such compounds.

with the proviso that there are no two adjacent ring oxygen atoms and no ring oxygen atom adjacent to either a ring nitrogen atom or a ring sulfur atom in any of the heterocyclic or heteroaryl moieties of formula I;

Preferred compounds of the formula I include those wherein R¹ is selected from the group consisting of cyclopropylmethyl, allyl, methyl, ethyl, isopropyl, phenylethyl, and 4-pyridyl methyl.

Other examples of preferred compounds of the formula I are those wherein R² is selected from the group consisting of N,N-diethyl amide, N,N-methylethyl amide, diethyl carbinol, dimethyl carbinol, 2-pyridine, 3-pyridine, 2-pyrimidine, and 2-thiazole.

Other examples of preferred compounds of the formula I are those wherein R³ is selected from the group consisting of methoxy, fluorine, amide, N-methyl amide, hydroxy, methylsulfonamide, and diethylsulfonamide.

The compounds of formula I and their pharmaceutically acceptable salts are opioid receptor ligands and are useful in the treatment of a variety of neurological and gastrointestinal disorders. Examples of disorders that can be treated with the compounds of formula I and their pharmaceutically acceptable salts are rejection in organ transplants and skin grafts, epilepsy, chronic pain, neurogenic pain, nonsomatic pain, stroke, cerebral ischemica, shock, head trauma, spinal cord trauma, brain edema, Hodgkin's disease, Sjogren's disease, systemic lupus erythematosis, gastrointestinal disorders such as gastritis, functional bowel disease, irritable bowel syndrome, functional diarrhoea, functional distention, nonulcerogenic dyspepsia and other disorders of motility or secretion, and emesis, acute pain, chronic pain, neurogenic pain, nonsomatic pain, allergies, respiratory disorders such as asthma, cough and apnea, inflammatory disorders such as rheumatoid arthritis, osteoarthritis, psoriasis and inflammatory bowel disease, urogenital tract disorders such as urinary incontinence, hypoxia (e.g., perinatal hypoxia), hypoglycemic neuronal damage, chemical dependencies and addictions (e.g., a dependency on, or addiction to opiates, benzodiazepines, cocaine, nicotine or ethanol), drug or alcohol withdrawal symptoms, and cerebral deficits subsequent to cardiac bypass surgery and grafting.

The present invention also relates to the pharmaceutically acceptable acid addition and base addition salts of compounds of the formula I. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)]salts. The chemical bases that are used as reagents to prepare the pharmaceutically acceptable base salts of this invention are those which form non-toxic base salts with the acidic compounds of formula I. Such non-toxic base salts include those derived from such pharmacologically acceptable cations as sodium, potassium, calcium and magnesium, etc.

Examples of preferred compounds of the formula I are the following:

4-[(8-allyl-8-aza-bicyclo[3.2.1]oct-3-ylidene)-(3-methoxy-phenyl)-methyl]-N,N-diethyl-benzamide;

4-[(8-cyclopropylmethyl-8-aza-bicyclo[3.2.1]oct-3-ylidene)-(3-methoxy-phenyl)-methyl]-N,N-diethyl-benzamide;

4-[(8-aza-bicyclo[3.2.1]oct-3-ylidene)-(3-hydroxy-phenyl)-methyl]-N,N-diethyl-benzamide;

4-[(8-allyl-8-aza-bicyclo[3.2.1]oct-3-ylidene)-(3-hydroxy-phenyl)-methyl]-N,N-diethyl-benzamide;

4-[(8-cyclopropylmethyl-8-aza-bicyclo[3.2.1]oct-3-ylidene)-(3-hydroxy-phenyl)-methyl]-N,N-diethyl-benzamide;

4-[(8-phenylethyl-8-aza-bicyclo[3.2.1]oct-3-ylidene)-(3-hydroxy-phenyl)-methyl]-N,N-diethyl-benzamide;

4-[(8-4-pyridylmethyl-8-aza-bicyclo[3.2.1]oct-3-ylidene)-(3-hydroxy-phenyl)-methyl]-N,N-diethyl-benzamide;

4-[(8-ethyl-8-aza-bicyclo[3.2.1]oct-3-ylidene)-(3-hydroxy-phenyl)-methyl]-N,N-diethyl-benzamide;

4-[(8-allyl-8-aza-bicyclo[3.2.1]oct-3-ylidene)-(3-fluorophenyl)-methyl]-N,N-diethyl-benzamide;

4-[(8-allyl-8-aza-bicyclo[3.2.1]oct-3-ylidene)-(3-carboxamide)-methyl]-N,N-diethyl-benzamide;

4-[(8-allyl-8-aza-bicyclo[3.2.1]oct-3-ylidene)-(3-diethylcarbinol)-methyl]-N,N-diethyl-benzamide;

N,N-diethyl-4-[(3-hydroxy-phenyl)-(8-methyl-8-aza-bicyclo[3.2.1]oct-3-ylidene)-methyl]-benzamide; and

N,N-diethyl-4-{(3-hydroxy-phenyl)-[8-( 1-methyl-1H-pyrrol-2-ylmethyl)-8-aza-bicyclo[3.2.1]oct-3-ylidene]-methyl}-benzamide.

The present invention also relates to the pharmaceutically acceptable base addition salts of compounds of the formula I. These salts are all prepared by conventional techniques. The chemical bases that are used as reagents to prepare the pharmaceutically acceptable base salts of this invention are those which form non-toxic base salts with the acidic compounds of formula I. Such non-toxic base salts include those derived from such pharmacologically acceptable cations as sodium, potassium, calcium and magnesium.

For a review on pharmaceutically acceptable salts, see Berge et al., J. Pharm. Sci., 66, 1-19 (1977).

This invention also relates to a pharmaceutical composition for treating a disorder or condition, the treatment or prevention of which can be effected or facilitated by modulating (i.e., increasing or decreasing) binding to opioid receptors in a mammal, including a human, comprising an amount of a compound of the formula I, or a pharmaceutically effective salt thereof, that is effective in treating such disorder or condition and a pharmaceutically acceptable carrier.

This invention also relates to a method of treating a disorder or condition, the treatment of which can be effected or facilitated by modulating binding to opioid receptors in a mammal, comprising administering to a mammal in need of such treatment an amount of a compound of the formula I, or a pharmaceutically effective salt thereof, that is effective in treating such disorder or condition.

This invention also relates to a pharmaceutical composition for treating a disorder or condition selected from inflammatory diseases such as arthritis (e.g., rheumatoid arthritis and osteoarthritis), psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions (e.g., addictions to or dependencies on alcohol, opiates, benzodiazepines, nicotine, heroin or cocaine), chronic pain, nonsomatic pain, acute pain and neurogenic pain, systemic lupus erythematosis, Hodgkin's disease, Sjogren's disease, epilepsy and rejection in organ transplants and skin grafts in a mammal, including a human, comprising a glutamate neurotransmission modulating effective amount of a compound of the formula I, or a pharmaceutically salt thereof, and a pharmaceutically acceptable carrier.

This invention also relates to a method for treating a condition selected from inflammatory diseases such as arthritis, psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions (e.g., addictions to or dependencies on alcohol, opiates, benzodiazepines, nicotine, heroin or cocaine), chronic pain, nonsomatic pain, acute pain and neurogenic pain, systemic lupus erythematosis, Hodgkin's disease, Sjogren's disease, epilepsy and rejection in organ transplants and skin grafts, in a mammal, comprising administering to such mammal, including a human, an opioid receptor binding modulating effective amount of a compound of the formula I, or a pharmaceutically acceptable salt thereof.

This invention also relates to a pharmaceutical composition for treating a disorder or condition, the treatment of which can be effected or facilitated by modulating binding to opioid receptors in a mammal, including a human, comprising an opioid receptor binding modulating effective amount of a compound of the formula I, or a pharmaceutically acceptable salt thereof, and a pharmaceutically acceptable carrier.

This invention also relates to a method for treating a disorder or condition, the treatment of which can be effected or facilitated by modulating in a mammal, including a human, comprising administering to such mammal an opioid receptor binding modulating effective amount of a compound of the formula I or a pharmaceutically acceptable salt thereof.

This invention also relates to a method of treating a condition selected from inflammatory diseases such as arthritis, psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions (e.g., addictions to or dependencies on alcohol, opiates, benzodiazepines, nicotine, heroin or cocaine), chronic pain, nonsomatic pain, acute pain and neurogenic pain, systemic lupus erythematosis, Hodgkin's disease, Sjogren's disease, epilepsy and rejection in organ transplants and skin grafts in a mammal, comprising administering to a mammal in need of such treatment an amount of a compound of the formula I that is effective in treating such condition.

This invention also relates to a pharmaceutical composition for treating a condition selected from inflammatory diseases such as arthritis, psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions (e.g., addictions to or dependencies on alcohol, opiates, benzodiazepines, nicotine, heroin or cocaine), chronic pain, nonsomatic pain, acute pain and neurogenic pain, systemic lupus erythematosis, Hodgkin's disease, Sjogren's disease, epilepsy and rejection in organ transplants and skin grafts in a mammal, comprising an amount of a compound of the formula I that is effective in treating such condition and a pharmaceutically acceptable carrier.

Unless otherwise indicated, the alkyl groups referred to herein, as well as the alkyl moieties of other groups referred to herein (e.g., alkoxy), may be linear or branched, and they may also be cyclic (e.g., cyclopropyl, cyclobutyl, cyclopentyl, or cyclohexyl) or be linear or branched and contain cyclic moieties.

The term “alkoxy”, as used herein, means “—O-alkyl”, wherein “alkyl” is defined as above.

The term “alkylene”, as used herein, means an alkyl group having two available binding sites (i.e., -alkyl-, wherein alkyl is defined as above).

The term “treating” as used herein, refers to reversing, alleviating, inhibiting the progress of, or preventing the disorder or condition to which such term applies, or one or more symptoms of such disorder or condition. The term “treatment”, as used herein, refers to the act of treating, as “treating” is defined immediately above.

Unless otherwise indicated, “halo” and “halogen”, as used herein, refer to fluorine, bromine, chlorine or iodine.

Compounds of the formula I may have chiral centers and therefore may exist in different enantiomeric and diastereomic forms. This invention relates to all optical isomers and all other stereoisomers of compounds of the formula I, and to all racemic and other mixtures thereof, and to all pharmaceutical compositions and methods of treatment defined above that contain or employ such isomers or mixtures.

Formula I above includes compounds identical to those depicted but for the fact that one or more hydrogen or carbon atoms are replaced by isotopes thereof. Such compounds are useful as research and diagnostic tools in metabolism pharmokinetic studies and in binding assays. Specific applications in research include radioligand binding assays, autoradiography studies and in vivo binding studies.

### DETAILED DESCRIPTION OF THE INVENTION

The compounds of formula I can be prepared according to the methods illustrated in Schemes 1-9 and discussed below. In the reaction schemes and discussion that follow, unless otherwise indicated, R¹, R², and R³ and structural formula I are defined as above.

Scheme 1 illustrates a method for the preparation of compounds with the general formula I wherein R³ is (C₁-C₆)alkoxy or fluorine, R² is CONR⁵R⁶ and R¹ is as defined above with the proviso that it is not attached to the piperidine nitrogen at a secondary alkyl carbon or an aryl group. Referring to Scheme 1, a bromobenzene derivative of formula 0, wherein R³ is methoxy or fluorine, is cooled to −70° C. in dry tetrahydrofuran, and then a solution of n-butyllithium is added to it. The resulting solution is then treated with cyano tropane 2, which is produced in one step from N-benzyltropinone 1 and the solution is allowed to warm to room temperature. Subsequent acid hydrolysis of the crude mixture yields the corresponding compound of formula 3.

The compound of formula 3, in tetrahydrofuran at −70° C. is then treated with the product of the reaction of n-BuLi and compound 4 and the resulting solution is stirred at a temperature ranging from about −70° C. to the room temperature, to produce following acid hydrolysis the corresponding olefin derivative of formula 5. The compound of formula 5 is then treated with trifluoromethane sulfonic anhydride or another suitable reagent such as N-phenyltrifluoromethanesulfonimide, in the presence of a base such as pyridine, triethylamine, another trialkyl amine, an alkali metal hydride or an alkali metal carbonate, to form the trifluoromethane sulfonate ester of formula 6. This reaction is typically performed in dichloromethane at a temperature ranging from about 0° C. to the reflux temperature, preferably at about room temperature.

The compound of formula 6 is placed under a carbon monoxide atmosphere at a pressure ranging from about 14 to 100 psi, in a solution of dimethylsulfoxide and a lower alkanol such as methanol or ethanol, with a suitable trialkylamine base (e.g., triethylamine) and palladium acetate with 1,3-bis(diphenylphosphino)propane (DPPP) or another suitable palladium ligand to afford ester 7. Other suitable palladium catalysts such as bis(triphenylphosphine) palladium dichloride may also be used. This reaction is performed at temperatures ranging from about 20° C. to 100° C. to.

Treatment of the ester of formula 7 with an aluminum amide of a primary or secondary amine, for example, diethyl amine, in a solvent such as dichloroethane or toluene, at a temperature ranging from about 20° C. to about the reflux temperature, preferably at about the reflux temperature, yields the corresponding amide of formula 8. Variations in the nature of the R¹ group on the piperidine nitrogen can be effected in the following manner, as illustrated by process steps (8→9→10) in Scheme 1. The compound of formula 8 is placed under a hydrogen atmosphere at pressures ranging from about 14 to 100 psi, in ethanol or other another solvent such as acetic acid or methanol, to produce the corresponding compound of formula 9. This reaction is typically carried out at a temperature from about 0° C. to about the reflux temperature, preferably at about room temperature.

Treatment of the compound of formula 9 with an aldehyde and sodium triacetoxyborohydride or another reducing agent (e.g., sodium borohydride or sodium cyanoborohydride), in dichloromethane, 1,2 dichloroethane or another suitable solvent such as methanol, ethanol or toluene, at a temperature ranging from about 0° C. to 100° C., preferably at about room temperature, yields the desired compound of formula 10.

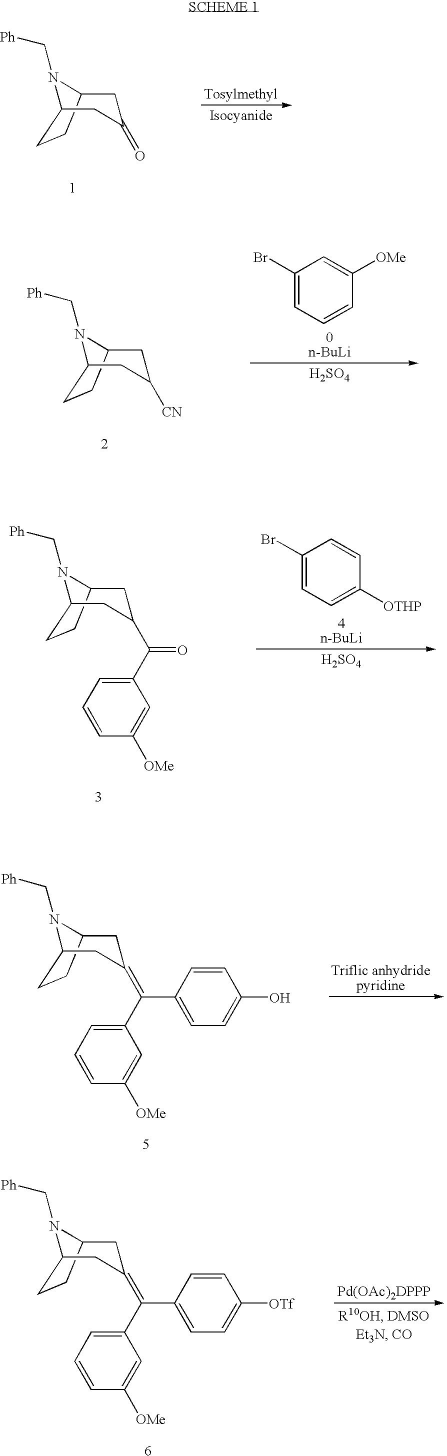

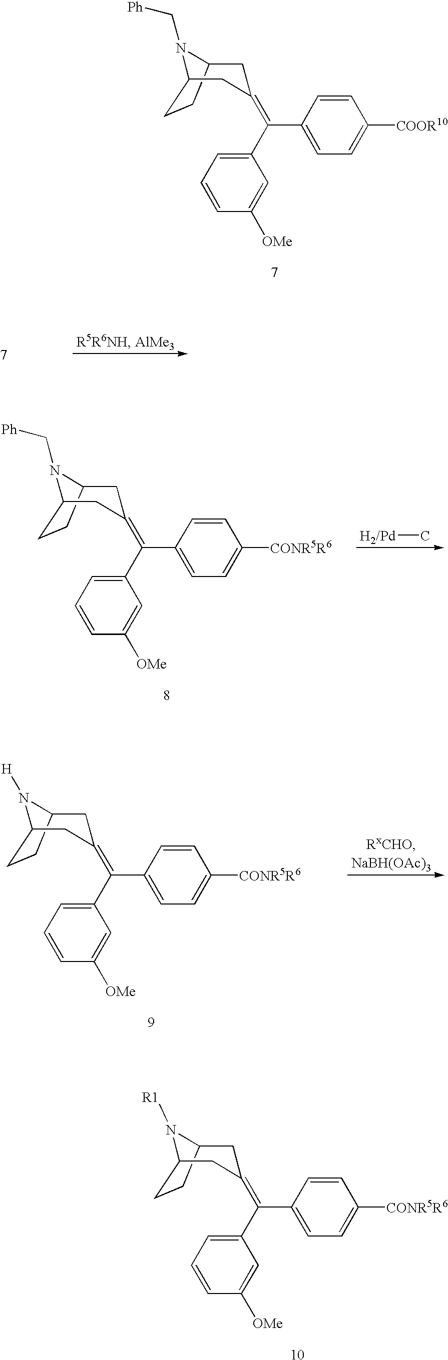

wherein R¹ is R^{x}CH₂—.

Alternatively, compounds of formula I where R3=OH can be prepared by the route described in Scheme la (compound 13). Treatment of the aryl halide 11 with n-BuLi at temperatures preferably ranging between −90° C. and −100° C. in THF as solvent followed by addition of a solution of ketone 3 in THF afforded carbinol of the formula 12. Treatment of 12 with acetic acid/aqueous HBr combination at temperatures ranging from room temperature to 120° C. afforded compound 13. Compound 13 can be debenzylated as shown in Scheme 1 and can be functionalized with conditions employed for the transformation of 9 to 10 to deliver compounds of formula 14 (Scheme 3) directly.

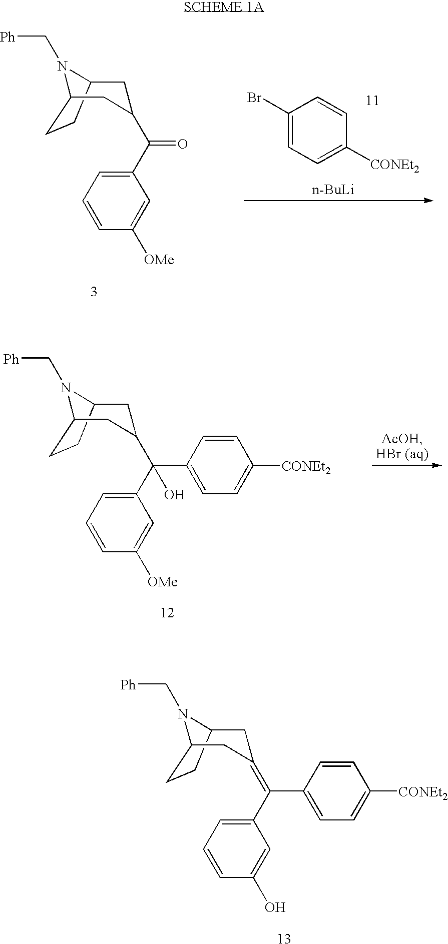

Compounds of formula I wherein R¹ is a group that attaches to the piperidine nitrogen via an aryl moiety or a primary or secondary alkyl moiety, can be prepared by treating the corresponding compound of formula 9 with an alkylating or arylating agent of the formula R¹X, wherein X is a leaving group such as chloro, bromo, iodo, triflate (OTf), mesylate (OMs) or tosylate (Ots), and sodium or potassium carbonate or another alkali metal carbonate or bicarbonate in a solvent such as dimethylformamide, dichloromethane or 1,2 dichloroethane, at a temperature ranging from about 20° C. to 100° C., as shown below in Scheme 2.

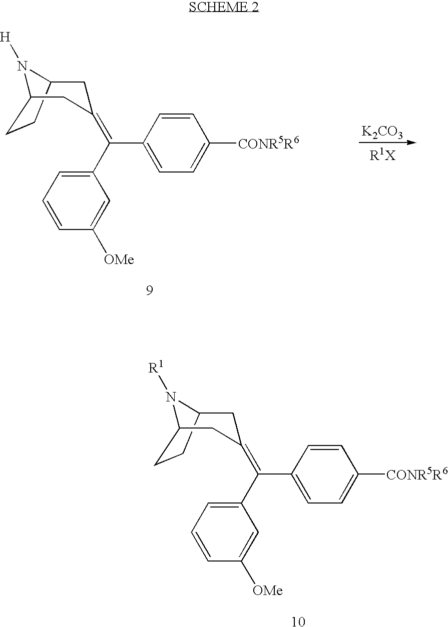

Compounds of the general formula I where R³ is hydroxy can be prepared by deprotecting the corresponding alkyl ether of formula 10 (wherein R¹⁰ is (C₁-C₆)alkyl) with boron tribromide in dicloromethane, or with aqueous hydrobromic acid and acetic acid, or with sodium ethanethiolate in dimethylformamide, at a temperature ranging from about 0° C. to the reflux temperature, as shown in Scheme 3. Room temperature is preferred when boron tribromide is used, the reflux temperature is preferred when hydrobromic acid/acetic acid is used, and about 100° C. to about 120° C. is preferred when sodium ethanemethiolate is used.

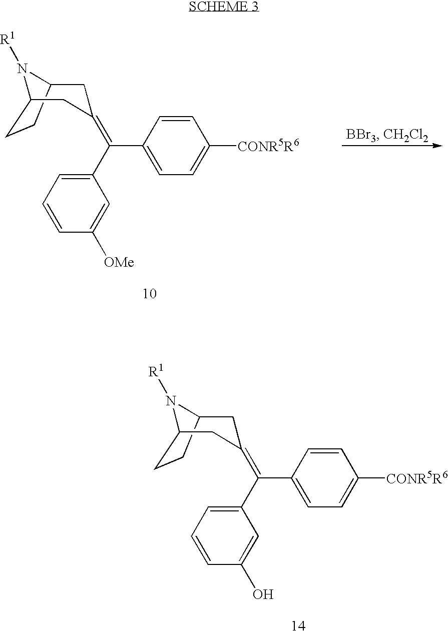

Compounds of the general formula I where R³=CONHR can be prepared from the corresponding phenols of formula 14 as illustrated in Scheme 4 below. This can be accomplished by formation of the triflate of formula 15 using conditions identical to those used for the preparation of compounds of the formula 6 (Scheme 1). The compound of formula 15 is then converted to the corresponding ester of formula 16 using conditions identical to those used in the preparation of esters of the formula 7 (Scheme 1). Treatment of the compound of formula 16 with an aluminum amide of an amine in a solvent such as toluene or 1,2 dichloroethane, at a temperature ranging from about 0° C. to about the reflux temperature, preferably at about the reflux temperature, or treatment of the same with a lithium amide in ether or tetrahydrofuran at a temperature ranging from about −78° C. to the reflux temperature, preferably at about −78° C., yields the desired compound of formula I wherein R³ is —CONHR⁴ and R⁴ is (formula 17 below).

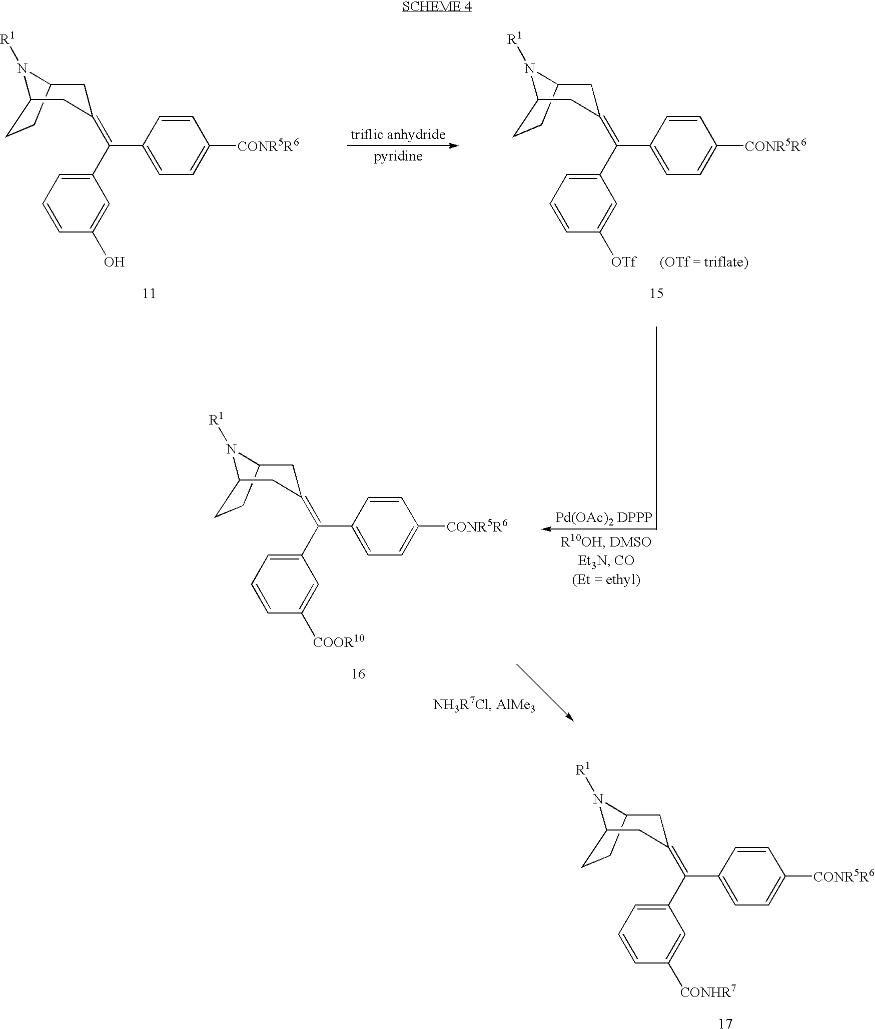

Alternatively, the carboxamide of formula 17 can be obtained by conversion of the triflate ester of formula 15 into the nitrile of formula 18 by treatment with zinc cyanide and a palladium catalyst such as tetrakis triphenylphosphine palladium, in a solvent such as dimethylformamide, or toluene, at a temperature from about 0° C. to about the reflux temperature, preferably at about the reflux temperature. The nitrile of formula 18 can be converted into the carboxamide of formula 17 by treatment with hydrogen peroxide and sodium carbonate in ethanol, at a temperature ranging from about 0° C. to about the reflux temperature, preferably at about room temperature.

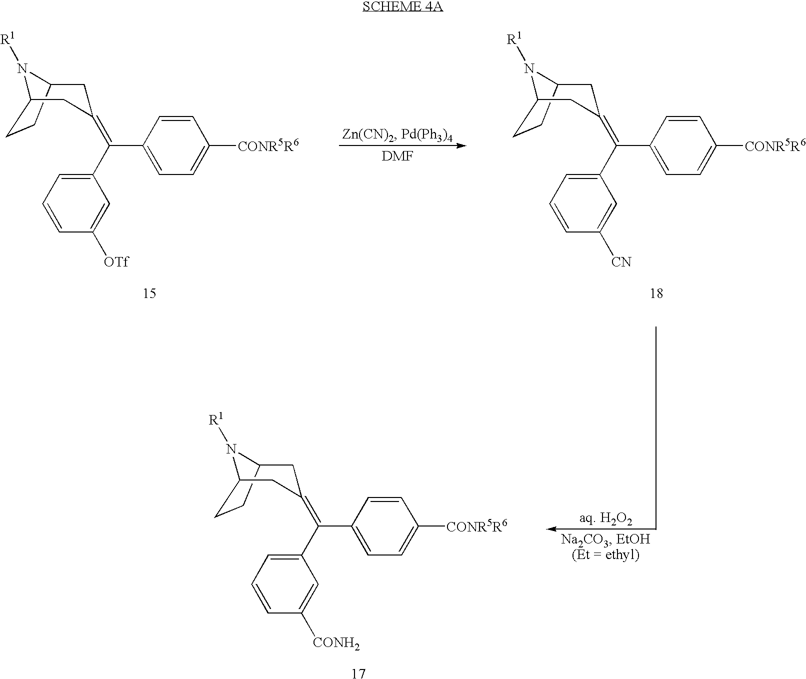

Compounds of the general formula I where R³ is NHSO₂R⁵ can be prepared, as illustrated in Scheme 5, by hydrolysis of the ester of formula 16 to the carboxylic acid of formula 19 by reacting it with lithium hydroxide or another alkali metal hydroxide in a mixture of tetrahydrofuran (THF) and water, at a temperature from about room temperature to about the reflux temperature. The compound of formula 19 is then converted into the aniline of formula 20 by reaction with diphenylphosphoryl azide in the presence of triethylamine or another trialkylamine base, in t-butanol at the reflux temperature, followed by acid hydrolysis with aqueous hydrochloric acid in ethyl acetate, or with trifluoroacetic acid in methylene chloride. The compound of the formula 20 is then sulfonylated to produce the desired compound of formula 21 with an alkyl- or arylsulfonyl chloride and pyridine triethylamine or another trialkylamine base in dichloromethane, dichloroethane or toluene, at temperatures from about 0° C. to about the reflux temperature, preferably at about room temperature.

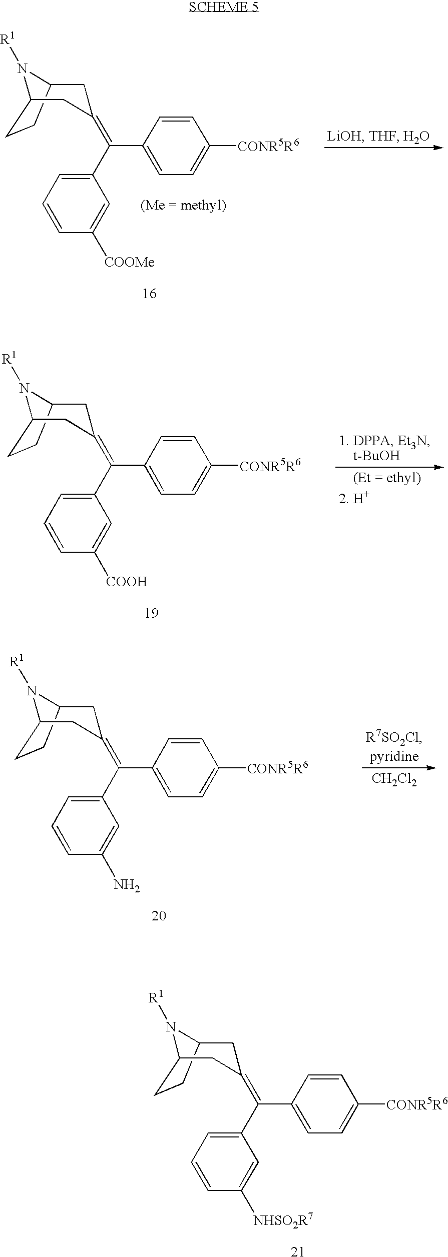

Compounds of the general formula I wherein R³ is methoxy, hydroxy or fluorine and R² is an aromatic or heteroaromatic moiety (referred to in Scheme 6 as compounds of the formula 22) can be prepared by organometalic coupling of a compound of the formula 6 with an aryl and heteroaryl boronic acid, wherein aryl and heteroaryl are defined as in the definitions of R¹ and R², in a solvent such as ethanol or toluene, in the presence of a palladium catalyst such as tetrakis triphenylphosphine palladium and a trialkylamine base (e.g., triethylamine) or alkali metal carbonate base, as shown below in Scheme 6. This reaction is generally carried out at a temperature from about room temperature to about the reflux temperature, preferably at about the reflux temperature.

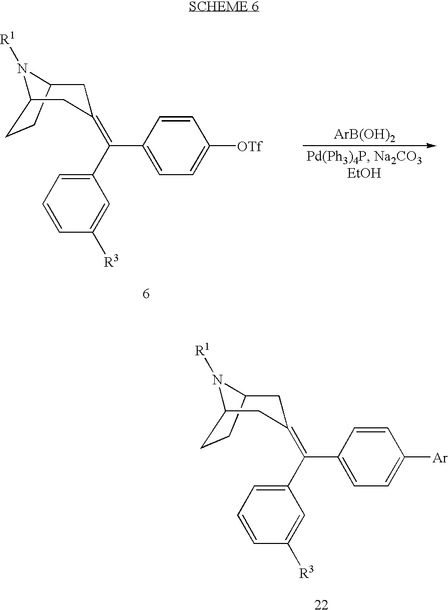

Compounds of the formula I wherein R² is tetrazoyl can be prepared, as illustrated in Scheme 7 below, by conversion of the appropriate triflate of formula 6 into the corresponding nitrile of formula 23. This can be accomplished by reacting the triflate compound with zinc cyanide and a palladium catalyst such as tetrakis triphenylphosphine palladium in a solvent such as dimethylformamide, at a temperature ranging from about 0° C. to about 100° C., preferably at about the reflux temperature. The formation of the tetrazole 24 proceeds by treatment of the resulting nitrile with sodium or trimethylsilylazide and a catalytic amount of tin oxide in a solvent such as dimethylformamide, preferably at about the reflux temperature or toluene, at a temperature ranging from about 20° C. to about the reflux temperature. Alkylation of the tetrazole to produce 25 proceeds by reaction with triethylamine or another trialkylamine base or an alkali metal hydride, alkoxide or carbonate, and with the appropriate compound of the formula R⁶X wherein X is a leaving group such as chloro, bromo, iodo, triflate, mesylate or tosylate, in a solvent such as methanol, ethanol, or tetrahydrofuran, at temperatures ranging from about 0° C. to about the reflux temperature, preferably at about room temperature.

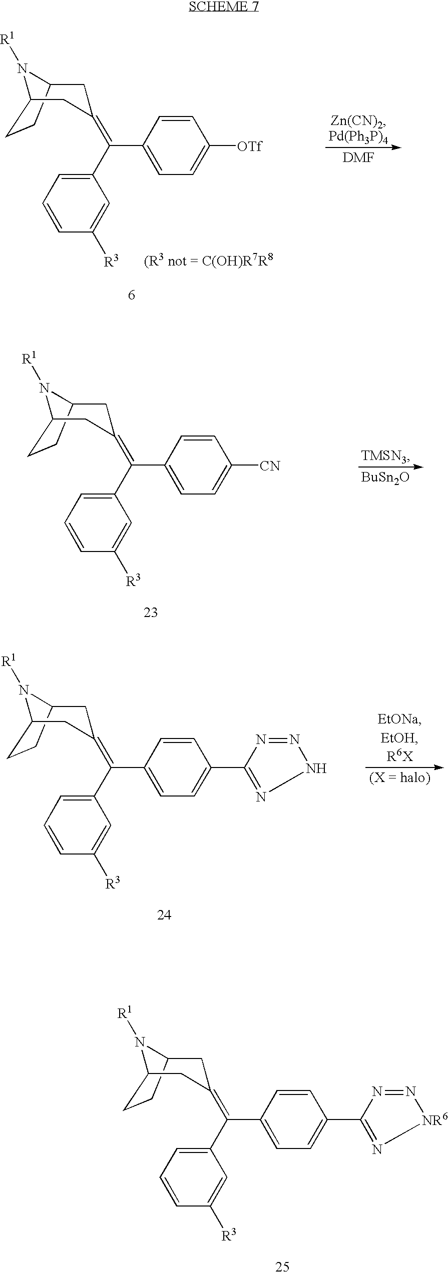

Compounds of the general formula I where R³ is fluoro or methoxy and R² is a carbinol such as diethyl carbinol (referred to in Scheme 9 as compounds of the formula 26) can be prepared, as illustrated in Scheme 9, by treatment of the ester of formula 7 with an alkyl Grignard or alkyl lithium reagent, in a solvent such as ether or tetrahydrofuran, at a temperature ranging from about −78° C. to about the reflux temperature, preferably starting at room temperature and heating to about the reflux temperature.

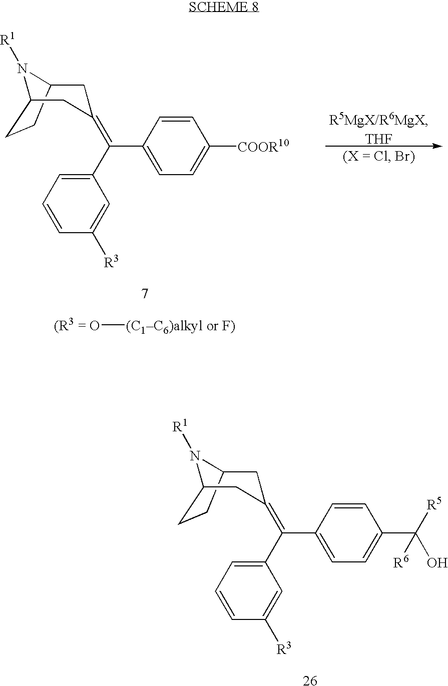

Compounds of the general formula I where R² is a diazaoxazole ring (e.g., compounds of the formula 29 in Scheme 10) can be prepared, as illustrated in Scheme 10, by treatment of the methyl ester of formula 7 with hydrazine hydrate in methanol, at a temperature from about 0° C. to about the reflux temperature, preferably at about the reflux temperature, to form the hydrazide of formula 27 Subsequent acylation with an acid chloride and pyridine, triethylamine or another trialkylamine in a solvent such as dichloromethane, dichloroethane or toluene, at a temperature from about 0° C. to about the reflux temperature, preferably at about room temperature provides the corresponding compound of formula 28. Cyclization can be accomplished using a reagent combination such as triphenylphospine/iodine and triethylamine or another trialkylamine in a solvent such as tetrahydrofuran, or toluene, at a temperature from about 0° C. to about the reflux temperature, preferably at about room temperature or using triflic anhydride and pyridine or a trialkylamine in dichloromethane, or tetrahydrofuran, at a temperature from about −78° C. to about room temperature, preferably starting at −78° C. and gradually warming to room temperature, or using thionyl chloride in dichloromethane, or neat, at a temperature from about room temperature to about the reflux temperature, preferably at about the reflux temperature, to yield the desired compound of formula 29.

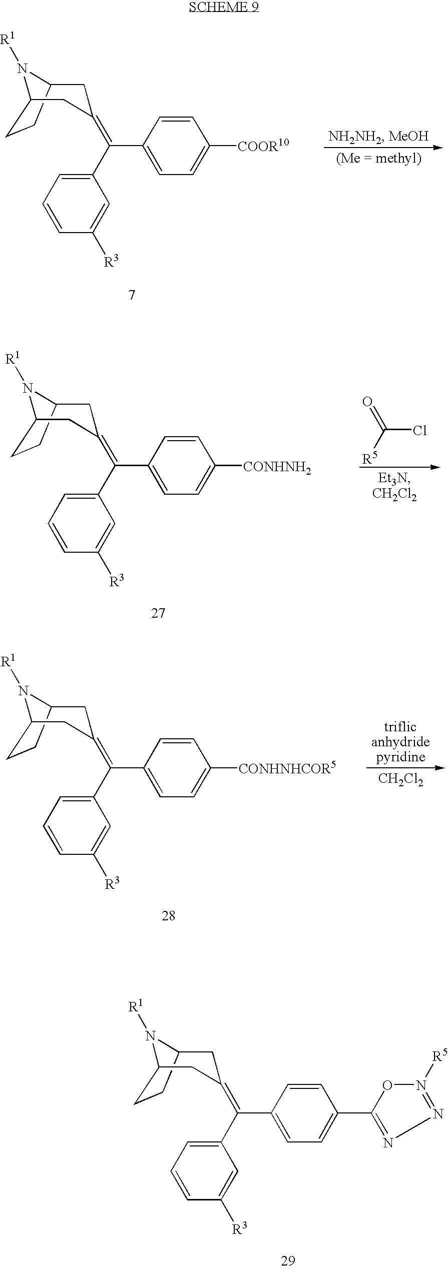

The preferred method of making compounds of the formula I wherein R³ is —OH, —NHSO₂R⁷, —C(OH)R⁷R⁸ or —C(═O)NHR⁷ is to make the analogous compounds wherein R³ is —O-(C₁-C₆)alkyl and then derivatize them using standards methods well known in art and illustrated in the foregoing schemes.

The starting materials used in the processes of Schemes 1-10 are either commercially available, known in the literature, or readily obtainable from commercially available or known compounds using methods that are well known in the art or described above.

Unless indicated otherwise, the pressure of each of the above reactions is not critical. Generally, the reactions will be conducted at a pressure from about one to about three atmospheres, preferably at ambient pressure (about one atmosphere).

The preparation of other compounds of the formula I not specifically described in the foregoing experimental section can be accomplished using combinations of the reactions described above that will be apparent to those skilled in the art.

The compounds of the formula I that are basic in nature are capable of forming a wide variety of different salts with various inorganic and organic acids. The acid that can be used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate [i.e., 1,1′-methylene-bis-(2-hydroxy-3-naphthoate)] salts. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Compounds of the formula that are acidic in nature are capable of forming base salts with various pharmacologically acceptable cations. These salts are all prepared by conventional techniques. The chemical bases that are used as reagents to prepare the pharmaceutically acceptable base salts of this invention are those which form non-toxic base salts with the acidic compounds of formula I. Such non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the formula I and the pharmaceutically acceptable salts thereof (hereinafter, also referred to, collectively, as “the active compounds of the invention”) are useful for the treatment of neurodegenerative, psychotropic and drug or alcohol induced deficits and are potent opioid receptor ligands. The active compounds of the invention may therefore be used in the treatment of disorders and conditions, such as those enumerated above, that can be treated by modulatiing binding to an opioid receptor.

The ability of the compounds of formula I to bind to the various opioid receptors and their functional activity at such receptors can be determined as described below. Binding to the delta opioid receptor can be determined using procedures well known in the art, such as those referred to by Lei Fang et al., J. Pharm. Exp. Ther., 268, 1994, 836-846 and Contreras et al., Brain Research, 604, 1993, 160-164.

In the description of binding and functional assays that follows, the following abbreviations and terminology are used.

DAMGO is [D-Ala2,N-MePhe4,Gly5-ol]enkephalin).

U69593 is ((5a, 7a, 8b)-(+)-N-methyl-N-(7-[1-pyrrolidinyl]-1-oxasipro[4,5]dec-8-yl)-benzeneacetamide).

SNC-80 is (+)-4-[(αR)-α((2S,5R)-4-allyl-2,5-dimethyl-1-piperazinyl)-3-methoxybenzyl]-N,N-diethylbenzamide.

nor BNI is nor-binaltorphimine.

CTOP is 1,2-Dithia-5,8,11,14,17-pentaazacycloeicosane, cyclic peptide derivative DPDPE is [D-en2,D-Pen5]enkephalin).

[3H]-DAMGO, [3H]-U69593, norBNI, and CTOP are all commercially available from DuPont, Amersham International, RBI and DuPont, Amersham International, RBI and DuPont respectively.

[3H]-SNC80 was prepared by Amersham International.

Opioid (mu and kappa) receptor binding assays can be performed in guinea-pig brain membrane preparations. Binding assays can be carried out at 25° C. for 60 minutes in 50 mM Tris (pH 7.4) buffer. [³H]-DAMGO(2 nM) and [³H]-U-69,593 (2 nM) can be used to label mu and kappa receptor binding sites, respectively. The protein concentration can be approximately 200 μg/well. Non-specific binding can be defined with 10 μM naloxone.

Delta receptor binding assays can be performed in a stable line of CHO cells expressing the human delta receptor. The binding assay can be carried out at 25° C. for 120 minutes in 50 mM Tris (pH 7.4) buffer. [³H]-SNC-80 can be used to label delta receptor binding sites. The protein concentration can be approximately 12.5 μg/well. Non-specific binding can be defined with 10 μM naltrexone.

The binding reaction can be terminated by rapid filtration through glass fibre filters, and the samples can be washed with ice-cold 50 mM Tris buffer (pH 7.4).

Agonist activity at the delta, mu and kappa opioid receptors can be determined as follows.

Opioid (delta, mu and kappa) activity is studied, as described below, in two isolated tissues, the mouse deferens (MVD)(δ) and the guinea-pig myentric plexus with attached longitudinal muscle (GPMP) (μ and k).

MVD (DC1 strain, Charles River, 25-35 g) are suspended in 15 ml organ baths containing Mg⁺⁺ free Krebs' buffer of the following composition (mM): NaCl, 119; KCl, 4.7; NaHCO₃, 25; KH₂PO₄, 1.2; CaCl₂, 2,5 and glucose, 11. The buffer is gassed with 95% 0₂ and 5% CO₂. The tissues are suspended between platinum electrodes, attached to an isometric transducer with 500 mg tension and stimulated with 0.03 Hz pulses of 1-msec pulse-width at supramaximal voltage. IC₅₀ values are determined by the regression analysis of concentration-response curves for inhibition of electrically-induced contractions in the presence of 300 nM of the mu-selective antagonist CTOP. This test is a measure of δ agonism.

Guinea-pig (Porcellus strain, male, 450-500 g, Dunkin Hartley) myentric plexus with attached longitudinal muscle segments are suspended with 1 g of tension in Krebs' buffer and stimulated with 0.1 Hz pulses of 1-msec pulse-width at supramaximal voltage. Mu functional activity is determined in the presence of 10 nM nor-BNI with 1 μM of the mu selective agonist, DAMGO, added to the bath at the end of the experiment to define a maximal response. This test is a measure of mu agonism.

Kappa functional activity is determined in the presence of and 1 μM CTOP with 1 μM of the kappa selective agonist U-69,593 added at the end of the experiment to define a maximal response. All inhibitions of twitch height for test compounds are expressed as a percentage of the inhibition obtained with the standard agonist and the corresponding IC₅₀ values determined.

The following procedure can be used to determine the activity of the therapeutic agents of this invention as agonists and as antagonists of delta opioid receptors.

Cell Culture: Chinese hamster ovary cells expressing the human delta opioid receptor are passaged twice weekly in Hamis F-12 media with L-glutamine containing 10% fetal bovine serum and 450 μg/mL hygromycin. Cells are prepared for assays 3 days prior to the experiment. 15 mL of 0.05% trypsin/EDTA is added to a confluent triple flask, swirled and decanted to rinse. 15 mL of 0.05% trypsin/EDTA is again added, and the flask is placed into a 37C incubator for 2 minutes. Cells are removed from the flask by banking, and supernatant poured off into a 50 mL tube. 30 mL of media is then added to the flask to stop the action of the trypsin, and then decanted into the 50 mL tube. Tube is then centrifuged for 5 minutes at 1000 rpm, media decanted, and the pellet resuspended into 10 mL of media. Viability of the cells is assessed using trypan blue, the cells counted and plated out into 96 well poly-D-lysine coated plates at a density of 7,500 cells/well.

Antagonist Test Plate: Cells plated 3 days prior to assay are rinsed twice with PBS. The plates are placed into a 37C water bath. 50 μL of assay buffer (PBS, dextrose 1 mg/mL, 5 mM MgC12, 30 mM HEPES, 66.7 μg/mL of IBMX) is then added to designated wells. Fifty microliters of appropriate drug is then added to designated wells, and timed for 1 minute. Fifty microliters of 10 μM forskolin+0.4 nM DPDPE (final assay concentration is 5 μM forskolin, 0.2 nM DPDPE) is then added to appropriate wells, and timed for 15 minutes. The reaction is stopped by the addition of 10 μL of 6N perchloric acid to all wells. To neutralize, 13 μL of 5N KOH is added to all wells, and to stabilize 12 μL of 2M Tris, pH 7.4 is added to all wells. Mix by shaking on an orbital shaker for 10 minutes, and centrifuge at setting 7 for 10 minutes. Alliquot into 3H plate.

Agonist Test Plate: Cells plated 3 days prior to assay are rinsed twice with PBS. The plates are placed into a 37° C. water bath. Fifty microliters of assay buffer (PBS, dextrose 1 mg/mL, 5 mM MgCl₂, 30 mM HEPES, 66.7 μg/mL of IBMX) is then added to designated wells. Fifty microliters of appropriate drug+10 μM forskolin (final assay concentration is 5 μM forskolin) is then added to all wells, and timed for 15 minutes. The reaction is then stopped by the addition of 10 μL of 6N perchloric acid to all wells. To neutralize, 13μ of 5N KOH is added to all wells, and to stablize 12 μL of 2M Tris, pH 7.4 is added to all wells. Mix by shaking on an orbital shaker for 10 minutes, and centrifuge at setting 7 for 10 minutes. Alliquot into 3H plate.

Both test plates are placed into an Amersham 3H cAMP binding kit overnight, and harvested onto GF/B filters previously soaked in 0.5% PEI with a Skatron using 50 mM Tris HCl pH 7.4 at 4° C. Filtermats can be air-dried overnight then place in bags with 20 ml Betaplate scintillation cocktail and counted on a Betaplate counter for 60 sec per sample. Data can be analyzed using Excel.

The compositions of the present invention may be formulated in a conventional manner using one or more pharmaceutically acceptable carriers. Thus, the active compounds of the invention may be formulated for oral, buccal, transdermal (e.g., patch), intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycollate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

In general, a therapeutically effective daily oral or intravenous dose of the compounds of formula (I) and their salts is likely to range from 0.001 to 50 mg/kg body weight of the subject to be treated, preferably 0.1 to 20 mg/kg. The compounds of the formula (I) and their salts may also be administered by intravenous infusion, at a dose which is likely to range from 0.001-10 mg/kg/hr.

Tables or capsules of the compounds may be administered singly or two or more at a time as appropriate. It is also possible to administer the compounds in sustained release formulations.

The physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

Alternatively, the compounds of the formula (I) can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stablisers and preservatives as may be required.

The following Examples illustrate the preparation of the compounds of the present invention. Commercial reagents were utilized without further purification. All NMR data were recorded at 250, 300 or 400 MHz in deuterochloroform unless otherwise specified and are reported in parts per million (δ) and are referenced to the deuterium lock signal from the sample solvent. All non-aqueous reactions were carried out in dry glassware with dry solvents under an inert atmosphere for convenience and to maximize yields. All reactions were stirred with a magnetic stirring bar unless otherwise stated. Unless otherwise stated, all mass spectra were obtained using chemical impact conditions. Ambient or room temperature refers to 20-25° C.

The following are further illustrative examples of the invention, through the invention is not limited to this descriptions therein

### EXAMPLE 1

### 8-Benzyl-8-aza-bicyclo[3.2.1]octane-3-carbonitrile

To a solution of n-benzyl tropinone (6.4 g) in DMF (200 mL) at room temperature was added TOSMIC (13.46 g). The reaction was thenstirred at room temperature for 30 minutes and was subsequently cooled to 0 C. Ethanol was then added (4.1 mL) followe by addition of potassium t-butixide (11.8 g) over 30 minutes through an addition funnel. The reaction mixture was allowed to warm to room temperature over the course of 2 hours and was then warmed to 60 C for 12 hours. The reaction was then allowed to cool to room temperature and was quenched by addition of brine (100 mL). The aqueous layer was washed with EtOAc (3×50 mL) and the combined organic extracts were dried over magnesium sulfate and concentrated under vacuum. Purification by flash chromatography afforded 8-Benzyl-8-aza-bicyclo[3.2.1]octane-3-carbonitrile (3.9 g). ¹HNMR (400 MHz, CDCl₃) δ 3.51 (s, 2H), 3.21 (s, 2H), 2.80-2.65 (m, 1H); MS (M+1)=227.

### EXAMPLE 2

### (8-Benzyl-8-aza-bicyclo[3.2.1]oct-3-yl)-(3-methoxy-phenyl)-methanone

To a solution of 3-bromoanisole (2.0 mL) in THF (30 mL) at −78 C was added a solution of n-BuLi (2.5 M in hexanes, 6.32 mL). The reaction mixture was stirred at −78 C for 1 hour. To the mixture was added a solution of compound 2 (3.6 g) in THF (20 mL). The reaction was aloowed to warm to room temperature over the course of 4 hours. The reaction mixture was then poured into a cold 30% aqueous solution of H₂SO4 (50 mL). The mixture was stirred vigorously for 20 minutes. The acid solution was washed once with diethyl ether (30 mL) and was subsequently brought to ph 10 with aqueous ammonium hydroxide. The basic water layer was extracted with ethyl acetate (3×50 mL). The.combined organic extracts were dried over magnesium sulfate and concentrated under vacuum. Purification by flash chromatography with hexanes/EtOAc (3:1) afforded ketone 3 (3.88 g). ¹HNMR (400 MHz, CDCl₃) δ 7.06 (d, 1H), 3.59 (s, 2H), 3.20 (s, 2H); MS (M+1)=336

### EXAMPLE 3

### 4-[(8-Benzyl-8-aza-bicyclo[3.2.1]oct-3-yl)-hydroxy-(3-methoxy-phenyl)-methyl]-N,N-diethyl-benzamide

To a solution of 4-bromo-N,N-diethyl-benzamide (3.16 g) in THF (20 mL) at −100 C was added n-BuLi (2.5M in hexanes, 4.9 mL) slowly so the internal temperature would not rise above −90 C. The mixture was stirred at −100 C for 15 minutes. To the reaction was added a solution of ketone 3 (2.75 g) in THF (10 mL) in one portion. The reaction mixture was stirred at −78 C for 1 hour and then was warmed to room temperature over the course of 3 hours. The mixture was poured into a saturated aqueous solution of sodium bicarbonate (30 mL). The aqueous layer was washed with EtOAc (3×30 mL) and the combined organic extracts were dried over magnesium sulfate and concentrated under vacuum. Purification by flash chromatography with methanol/EtOAc (1:10) afforded alcohol 12 (2.1 g). ¹HNMR (400 MHz, CDCl₃) δ 6.65 (d, 1H), 3.21 (s, 2H), 2.94-2.81 (m, 1H); MS (M+1)=513

### EXAMPLE 4

### 4-[(8-Benzyl-8-aza-bicyclo[3.2.1]oct-3-ylidene)-(3-hydroxy-phenyl)-methyl]-N,N-diethyl-benzamide

A solution of alcohol 12 (0.93 g) in glacial acetic acid (9 mL) and concentrated aqueous HBr (9 mL) was heated to reflux for 3 hours. The mixture was cooled to room temperature and was then slowly added to concentrated aqueous ammonium hydroxide (60 mL). The aqueous layer was washed with dichloromethane (2×20 mL). The combined organic extracts were dried over magnesium sulfate and concentrated under vacuum. Purification of the resulting residue by flash chromatography with dichloromethane/methanol (10:1) afforded the desired phenol 13 (0.78 g). ¹HNMR (400 MHz, CDCl₃) δ6.60 (d, 2H), 2.26 (d, 1H), 2.15 (d, 1H); MS (M+1)=481

### EXAMPLE 5

### 4-[(8-Aza-bicyclo[3.2.1]oct-3-ylidene)-(3-hydroxy-phenyl)-methyl]-N,N-diethyl-benzamide

The hydrochloride salt of olefin 8 (0.72 g) was dissolved in 20 ml ethanol and placed in a high pressure hydrogenation bottle. To the solution was added 10% palladium hydroxide on carbon (0.8 g) and the solution was shaken under 50 psi of hydrogen for 16 hours. The mixture was filtered through a plug of celite and the catalust cake was washed with additional ethanol (30 mL). The ethanol was removed under reduced pressure to afford 0.54 g of the desired amine hydrochloride (9, R³ is hydroxyl). ¹HNMR (400 MHz, CDCl₃) δ 7.16 (t, 1H), 6.58 (s, 1H), 4.02 (s, 2H); MS (M+1) 391.

### General Procedure for the Reductive Alkylation of 4-[(8-Aza-bicyclo[3.2.1]oct-3-ylidene)-(3-hydroxy-phenyl)-methyl]-N,N-diethyl-benzamide (Reductive Alkylation of Compound 9, R³ is Hydroxyl)

To a solution of the hydrochloride salt of 4-[(8-aza-bicyclo[3.2.1]oct-3-ylidene)-(3-hydroxy-phenyl)-methyl]-N,N-diethyl-benzamide (1 equivalent) in CH₂Cl₂ (0.4M) was added the aldehyde R^{x}CHO (1.2 equivalents) followed by addition of acetic acid (1.2 equivalents) and NaBH(OAc)₃ (1.5 equivalents). The reaction mixture was stirred at room temperature for 16 hours. The mixture was then partitioned between equal volumes of CH₂Cl₂ and sat. aqueous NaHCO₃. The organic layer was separated and the aqueous layer was washed with CH₂Cl₂ (3×). The combined organic layers were dried (MgSO₄) and concentrated. Purification by flash chromatography afforded the desired tertiary amines in yields ranging from 60-95%. Through the reaction of compounds of formula 9 (R³ is hydroxyl) with the appropriate aldehyde R^{x}CHO (as shown in the conversion of compound 9 into compound 10 at the end of Scheme 1) this procedure was used to prepare the title compounds of Examples 6 through 30.

### EXAMPLE 6

### N,N-Diethyl-4-{(3-hydroxy-phenyl)-[8-(3-phenyl-propyl)-8-aza-bicyclo[3.2.1]oct-3-ylidene]-methyl}-benzamide

¹HNMR (400 MHz, CDCl₃) δ 6.78-6.62 (comp, 2H), 3.69-3.62 (comp, 2H), 1.18-1.04 (comp, 3H); MS (M+1)=509

### EXAMPLE 7

### 4-[[8-(3-Chloro-benzyl)-8-aza-bicyclo[3.2.1]oct-3-ylidene]-(3-hydroxy-phenyl)-methyl]-N,N-diethyl-benzamide

¹HNMR (400 MHz, CDCl₃) δ 7.38 (s, 1H), 6.58-6.42 (comp, 3H), 2.04 (d, 1H); MS (M+1)=515

### EXAMPLE 8

### 4-[[8-(4-Chloro-benzyl)-8-aza-bicyclo[3.2.1]oct-3-ylidene]-(3-hydroxy-phenyl)-methyl]-N,N-diethyl-benzamide

¹HNMR (400 MHz, CDCl₃) δ 2.58-2.41 (m, 1H), 2.25 (d, 1H), 2.06 (d, 1H); MS (M+1)=515

### EXAMPLE 9

### N,N-Diethyl-4-[(3-hydroxy-phenyl)-(8-phenethyl-8-aza-bicyclo[3.2.1]oct-3-ylidene)-methyl]-benzamide

¹HNMR (400 MHz, CDCl₃) δ 6.65 (d, 1H), 2.31 (d, 1H), 2.20 (d, 1H); MS (M+1) 495

### EXAMPLE 10

### 4-[[8-(2-Chloro-benzyl)-8-aza-bicyclo[3.2.1]oct-3-ylidene]-(3-hydroxy-phenyl)-methyl]-N,N-diethyl-benzamide

¹HNMR (400 MHz, CDCl₃) δ 6.68-6.60 (comp, 2H), 6.48 (d, 1H), 2.38 (d, 1H); MS (M+1)=515

### EXAMPLE 11

### 4-[(8-Benzo[1,3]dioxol-5-ylmethyl-8-aza-bicyclo[3.2.1]oct-3-ylidene)-(3-hydroxy-phenyl)-methyl]-N,N-diethyl-benzamide

¹HNMR (400 MHz, CDCl₃) δ 7.39 (s, 1H), 6.59 (s, 2H), 2.43 (d, 1H); MS (M+1)=525

### EXAMPLE 12

### N,N-Diethyl-4-[(3-hydroxy-phenyl)-(8-methyl-8-aza-bicyclo[3.2.1]oct-3-ylidene)-methyl]-benzamide

¹HNMR (400 MHz, CDCl₃) δ 2.79-2.60 (comp, 2H), 2.42 (s, 3H), 2.46 (d, 1H); MS (M+1)=405

### EXAMPLE 13

### N,N-Diethyl-4-{(3-hydroxy-phenyl)-[8-(4-methyl-benzyl)-8-aza-bicyclo[3.2.1]oct-3-ylidene]-methyl}-benzamide

¹HNMR (400 MHz, CDCl₃) δ 3.18 (s, 2H), 2.29 (s, 3H), 2.06 (d, 1H); MS (M+1)=495

### EXAMPLE 14

### N,N-Diethyl-4-{(3-hydroxy-phenyl)-[8-(3-methyl-benzyl)-8-aza-bicyclo[3.2.1]oct-3-ylidene]-methyl}-benzamide

¹HNMR (400 MHz, CDCl₃) δ 6.57 (s, 1H), 3.24 (s, 2H), 2.29 (s, 3H); MS (M+1)=495

### EXAMPLE 15

### N,N-Diethyl-4-{(3-hydroxy-phenyl)-[8-(4-methoxy-benzyl )-8-aza-bicyclo[3.2.1]oct-3-ylidene]-methyl}-benzamide

¹HNMR (400 MHz, CDCl₃) δ 6.81 (d, 2H), 3.75 (s, 3H), 2.38-2.28 (m, 1H); MS (M+1)=511

### EXAMPLE 16

### N,N-Diethyl-4-[[8-(3-fluoro-benzyl)-8-aza-bicyclo[3.2.1]oct-3-ylidene]-(3-hydroxy-phenyl)-methyl]-benzamide

¹HNMR (400 MHz, CDCl₃) δ 6.96-6.88 (m, 1H), 2.24 (d, 1H), 1.63-16.0 (m, 1H); MS (M+1)=499

### EXAMPLE 17

### N,N-Diethyl-4-[[8-(4-fluoro-benzyl)-8-aza-bicyclo[3.2.1]oct-3-ylidene]-(3-hydroxy-phenyl)-methyl]-benzamide

¹HNMR (400 MHz, CDCl₃) δ 6.54 (s, 1H), 2.12 (d, 1H), 1.61-1.58 (m, 1H); MS (M+1)=499.

### EXAMPLE 18

### N,N-Diethyl-4-{(3-hydroxy-phenyl)-[8-(2-trifluoromethyl-benzyl)-8-aza-bicyclo[3.2.1]oct-3-ylidene]-methyl}-benzamide

¹HNMR (400 MHz, CDCl₃) δ 7.98 (br s, 1H), 3.24 (s, 2H), 2.16 (d, 1H); MS (M+1)=549

### EXAMPLE 19

### N,N-Diethyl-4-{(3-hydroxy-phenyl)-[8-(4-methoxy-3-methyl-benzyl )-8-aza-bicyclo[3.2.1]oct-3-ylidene]-methyl}-benzamide

¹HNMR (400 MHz, CDCl₃) δ 6.65 (d, 1H), 3.77 (s, 3H), 2.14 (s, 3H); MS (M+1)=525

### EXAMPLE 20

### N,N-Diethyl-4-{(3-hydroxy-phenyl)-[8-(4-methylsulfanyl-benzyl)-8-aza-bicyclo[3.2.1]oct-3-ylidene]-methyl}-benzamide

¹HNMR (400 MHz, CDCl₃) δ 2.42 (s, 3H), 2.24 (d, 1H), 2.17 (d, 1H); MS (M+1)=527

### EXAMPLE 21

### 4-[{8-[3-(4-Chloro-phenoxy)-benzyl]-8-aza-bicyclo[3.2.1]oct-3-ylidene}-(3-hydroxy-phenyl)-methyl]-N,N-diethyl-benzamide

¹HNMR (400 MHz, CDCl₃) δ 7.07 (d, 2H), 3.23 (s, 2H), 2.24 (d, 1H); MS (M+1)=607

### EXAMPLE 22

### N,N-Diethyl-4-{(3-hydroxy-phenyl)-[8-(4-phenoxy-benzyl)-8-aza-bicyclo[3.2.1]oct-3-ylidene]-methyl}-benzamide

¹HNMR (400 MHz, CDCl₃) δ 7.32 (m, 1H), 6.58 (s, 1H), 1.16 (d, 1H); MS (M+1)=573

### EXAMPLE 23

### N,N-Diethyl-4-{(3-hydroxy-phenyl)-[8-(4-isopropyl-benzyl)-8-aza-bicyclo[3.2.1]oct-3-ylidene]-methyl}-benzamide

¹HNMR (400 MHz, CDCl₃) δ 2.84 (m, 1H), 2.15 (d, 1H), 1.22 (d, 6H); MS (M+1 )=523

### EXAMPLE 24

### N,N-Diethyl-4-[(3-hydroxy-phenyl)-(8-thiophen-2-ylmethyl-8-aza-bicyclo[3.2.1]oct-3-ylidene)-methyl]-benzamide

¹HNMR (400 MHz, CDCl₃) δ 6.93 (s, 1H), 6.60 (s, 1H), 2.17 (d, 1H); MS (M+1)=487

### EXAMPLE 25

### N,N-Diethyl-4-{(3-hydroxy-phenyl)-[8-(1-methyl-1H-pyrrol-2-ylmethyl)-8-aza-bicyclo[3.2.1]oct-3-ylidene]-methyl}-benzamide

¹HNMR (400 MHz, CDCl₃) δ 7.71-7.69 (m, 1H), 7.53-7.51 (m, 1H), 2.17 (d, 1H); MS (M+1)=484

### EXAMPLE 26

### N,N-Diethyl-4-[(3-hydroxy-phenyl)-(8-quinolin-3-ylmethyl-8-aza-bicyclo[3.2.1]oct-3-ylidene)-methyl]-benzamide

¹HNMR (400 MHz, CDCl₃) δ 8.16 (d, 1H), 8.02 (d, 1H), 2.24 (d, 1H); MS (M+1)=532

### EXAMPLE 27

### N,N-Diethyl-4-[(8-furan-3-ylmethyl-8-aza-bicyclo[3.2.1]oct-3-ylidene)-(3-hydroxy-phenyl)-methyl]-benzamide

¹HNMR (400 MHz, CDCl₃) δ 7.36 (s, 1H), 2.29 (d, 1H), 2.19 (d, 1H); MS (M+1)=471

### EXAMPLE 28

### N,N-Diethyl-4-[(3-hydroxy-phenyl)-(8-quinolin-4-ylmethyl-8-aza-bicyclo[3.2.1]oct-3-ylidene)-methyl]-benzamide

¹HNMR (400 MHz, CDCl₃) δ 7.96 (δ, 1H), 6.61(σ, 1H), 2.27 (δ, 1H); MS (M+1)=532

### EXAMPLE 29

### 4-[(8-Cyclohex-3-enylmethyl-8-aza-bicyclo[3.2.1]oct-3-ylidene)-(3-hydroxy-phenyl)-methyl]-N,N-diethyl-benzamide

¹HNMR (400 MHz, CDCl₃) δ 5.65 (s, 2H), 3.54 (s, 2H), 1.57 (d, 1H); MS (M+1)=485

### EXAMPLE 30

### 2-{3-[(4-Diethylcarbamoyl-phenyl)-(3-hydroxy-phenyl)-methylene]-8-aza-bicyclo[3.2.1]oct-8-ylmethyl}-cyclopropanecarboxylic Acid Ethyl Ester

¹HNMR (400 MHz, CDCl₃) δ 2.30 (d, 1H), 2.17 (d, 1H), 0.91-0.81 (comp, 2H); MS (M+1)=517.

### General Procedure for the Alkylation of 4-[(8-Aza-bicyclo[3.2.1]oct-3-ylidene)-(3-methoxy-phenyl)-methyl]-N,N-diethyl-benzamide (Compounds of Scheme 2)

To a solution of N,N-diethyl-4-[4-(3-methoxy-phenyl)-piperidin-4-yl]-benzamide (1 equivalent) in DMF (0.5M) was added K₂CO₃ (3-10 equivalents) and the alkyl or heteroaryl halide (1-5 equivalents). The reaction mixture was stirred at 60-120° C. for 3-16 hours. The mixture was then cooled to room temperature and filtered. The filtrate was diluted with diethyl ether and the ether layer was washed with brine. The organic phase was dried (MgSO₄) and concentrated. Purification by flash chromatography afforded the desired amines in yields ranging from 30-85%. This procedure was used to prepare the title compounds of Examples 31 and 32.

### EXAMPLE 31

### 4-[(8-Allyl-8-aza-bicyclo[3.2.1]oct-3-ylidene)-(3-methoxy-phenyl)-methyl]-N,N-diethyl-benzamide (10a)

¹HNMR (400 MHz, CDCl₃) δ 6.63 (s, 1H), 6.38-6.26 (m, 1H), 5.43 (d, 1H); MS (M+1)=445.

### EXAMPLE 32

### 4-[(8-Cyclopropylmethyl-8-aza-bicyclo[3.2.1]oct-3-ylidene)-(3-methoxy-phenyl)-methyl]-N,N-diethyl-benzamide (10b)

¹HNMR (400 MHz, CDCl₃) δ 2.42 (d, 2H), 0.52 (d, 2H), 0.14 (d, 2H); MS (M+1)=459.

### EXAMPLE 33

### (8-Benzyl-8-aza-bicyclo[3.2.1]oct-3-yl)-(3-methoxy-phenyl)-[4-(tetrahydro-pyran-2-yloxy)-phenyl]-methanol (4)

To a solution of 4-(tetrahydro-pyran-2-yloxy)-phenyl bromide (6.3 g) in THF (60 mL) at −78 C was added a solution of n-BuLi (2.5M in hexanes, 9.8 mL). The reaction mixture was stirred at −78 C for 1 hour. To the reaction was added a solution of ketone 3 (8.22 g) in THF (40 mL). The reaction was stirred at −78 C for 1 hour and was allowed to warm to room temperature over the course of 3 hours. The reaction mixture was added to a saturated aqueous solution of sodium bicarbinate (40 mL). The aqueous layer was washed with EtOAc (3×40 mL) and the combined organic layers were dried (MgSO₄) and concentrated. Purification by flash chromatography afforded the tertiary alcohol 4 (7.4 g). ¹HNMR (400 MHz, CDCl₃) δ 6.65 (d, 1H), 5.33 (s, 1H), 3.75 (s, 3H); MS (M+1)=514

### EXAMPLE 34

### 4-[(8-Benzyl-8-aza-bicyclo[3.2.1]oct-3-ylidene)-(3-methoxy-phenyl)-methyl]-phenol (5)

A solution of alcohol in thionyl chloride (15 mL) was heated to reflux for 3 hours. The mixture was then concentrated and the resulting residue partitioned between a saturated aqueous solution of sodium bicarbonate (20 mL) and dichloromethane (20 mL). The aqueous layer was washed with dichloromethane (3×20 mL) and the combined organic layers were dried (MgSO₄) and concentrated. Purification by flash chromatography afforded the desired olefin 5 (1.8 g). ¹HNMR (400 MHz, CDCl₃) δ 7.82 (d, 1H), 6.58 (d, 1H), 6.55 (s, 1H); MS (M+1)=412

### EXAMPLE 35

### Trifluoro-methanesulfonic Acid 4-[(8-benzyl-8-aza-bicyclo[3.2.1]oct-3-ylidene)-(3-methoxy-phenyl)-methyl]-phenyl Ester (6)

A solution of phenol (0.63 g) in dichloromethane (5 mL) at 0 C was treated with pyridine (o.6 mL) and triflic anhydride (0.39 mL). The reaction mixture was stirred at 0 C for 3 hours. To the reaction mixture was added a saturated aqueous solution of sodium bicarbonate (5 mL) and the layers were separated. The aqueous layer was washed with dichloromethane (3×10 mL) and the combined organic layers were dried (MgSO₄) and concentrated. Purification by flash chromatography afforded the desired triflate 6 (0.60 g). ¹HNMR (400 MHz, CDCl₃) δ 6.58 (s, 1H), 2.26 (d, 1H), 2.15 (d, 1H); MS (M+1)=544

### General Procedure for the Coupling of Triflate and Aryl Boronic Acids (Compounds of Formula 22)

A solution of triflate (1 equivalent) in ethanol/water (9:1 ratio, 0.1M overall) was charged with palladium tetrakis triphenyl phosphine (0.1 equivalents), sodium carbonate (2.5 equivalents) and aryl boronic acid (1.5 equivalents). The reaction mixture was degassed and then was heated to 90 C for 16 hours. The reaction was then cooled to room temperature and concentrated. The resulting residue was purified by flash chromatography to afford the desired biaryl coupled products in yields ranging from 53-88%. This procedure was used to prepare the title compounds of Example 36 through 38.

### EXAMPLE 36

### 8-Benzyl-3-[(3′-chloro-4′-fluoro-biphenyl-4-yl)-(3-methoxy-phenyl)-methylene]-8-aza-bicyclo[3.2.1]octane

¹HNMR (400 MHz, CDCl₃) δ 6.75 (d, 1H), 6.69 (s, 1H), 2,38-2.24 (comp, 2H); MS (M+1)=524

### EXAMPLE 37

### 8-Benzyl-3-[(3-methoxy-phenyl)-(4-thiophen-2-yl-phenyl)-methylene]-8-aza-bicyclo[3.2.1]octane

¹HNMR (400 MHz, CDCl₃) δ 7.72-7.60 M, 1H), 3. 58 (s, 3H) 3.21 (s, 2H); MS (M+1)=478

### EXAMPLE 38

### 8-Benzyl-3-[(3-methoxy-phenyl)-(4′-trifluoromethyl-biphenyl-4-yl)-methylene]-8-aza-bicyclo[3.2.1]octane

¹HNMR (400 MHz, CDCl₃) δ 3.76 (s, 3H), 3.38 (s, 2H), 2.38-2.29 (comp, 2H); MS (M+1)=540

### General Procedure for the Deprotection of Methyl Ethers

(a) To a suspension of NaH (10 equivalents) in DMF (0.2M) at room temperature was added ethane thiol (10 equivalents) dropwise. The mixture was stirred for 5 minutes. To the reaction mixture was added a solution of the methyl ether (1 equivalent) in DMF (0.2M). The mixture was heated to 120° C. for 10-16 hours. The reaction was cooled to room temperature and was quenched with water. The mixture was diluted with diethyl ether and the organic layer was washed with brine. The organic phase was dried (MgSO₄) and concentrated. Purification by flash chromatography afforded the desired phenols in yields ranging from 30-95%.

(b) To a solution of methyl ether (1 equivalent) in CH₂Cl₂ (0.4M) at −78° C. was added a solution of boron tribromide (1-5 equivalents) in CH₂Cl₂ (1.0M) dropwise. The reaction mixture was stirred at −78° C. for 1 hour was warmed to room temperature and stirred for an additional 4-6 hour. The mixture was quenched with slow addition of water and was brought to pH 8 with a saturated water/NH₄OH solution. The aqueous layer was washed with CH₂Cl₂. The organic phase was dried (MgSO₄) and concentrated. Purification by flash chromatography afforded the desired phenols in yields ranging from 60-95%.

Compounds of Scheme 3

### EXAMPLE 39

### 4-[(8-Allyl-8-aza-bicyclo[3.2.1]oct-3-ylidene)-(3-hydroxy-phenyl)-methyl]-N,N-diethyl-benzamide

¹HNMR (400 MHz, CDCl₃) δ 6.65 (d, 1H), 6.38-6.59 (m, 1H), 5.35-5.28 (comp, 2H); MS (M+1)=431.

### EXAMPLE 40

### 4-[(8-Cyclopropylmethyl-8-aza-bicyclo[3.2.1]oct-3-ylidene)-(3-hydroxy-phenyl)-methyl]-N,N-diethyl-benzamide

¹HNMR (400 MHz, CDCl₃) δ 6.58 (d, 1H), 2.42 (d, 1H), 0.64-0.59 (comp, 2H); MS (M+1)=445

Deprotection of compounds from Scheme 6

### EXAMPLE 41

### 3-[(8-Benzyl-8-aza-bicyclo[3.2.1]oct-3-ylidene)-(3′-chloro-4′-fluoro-biphenyl-4-yl)-methyl]-phenol

¹HNMR (400 MHz, CDCl₃) δ 6.69 (d, 1H), 6.67 (s, 1H), 6.39 (d, 1H); MS (M+1)=510

### EXAMPLE 42

### 3-[(8-Benzyl-8-aza-bicyclo[3.2.1]oct-3-ylidene)-(4-thiophen-2-yl-phenyl)-methyl]-phenol

¹HNMR (400 MHz, CDCl₃) δ 6.63 (d, 1H), 3.39 (s, 2H), 2.56-2.42 (comp, 2H); MS (M+1)=464

### EXAMPLE 43

### 3-[(8-Benzyl-8-aza-bicyclo[3.2.1]oct-3-ylidene)-(4′-trifluoromethyl-biphenyl-4-yl)-methyl]-phenol

¹HNMR (400 MHz, CDCl₃) δ 3.42 (s, 2H), 2.41-2.28 (comp, 2H), 1.88-1.64 (comp, 2H)

1. A compound of the formula 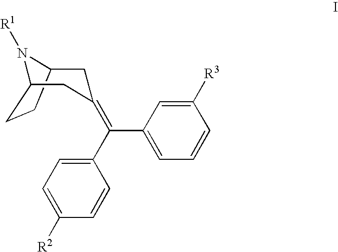
R¹ is hydrogen, (C₁-C₈)alkoxy-(C₁-C₈)alkyl-, wherein the total number of carbon atoms is eight or less, aryl, aryl-(C₁-C₈)alkyl-, heteroaryl, heteroaryl-(C₁-C₈)alkyl-, heterocyclic, heterocyclic-(C₁-C₈)alkyl, (C₃-C₇)cycloalkyl-, or (C₃-C₇)cycloalkyl-(C₁-C₈)alkyl, wherein said aryl and the aryl moiety of said aryl-(C₁-C₈)alkyl- are independently selected from phenyl and napthyl, and wherein said heteroaryl and the heteroaryl moiety of said heteroaryl-(C₁-C₈)alkyl- are independently selected from pyrazinyl, benzofuranyl, quinolyl, isoquinolyl, benzothienyl, isobenzofuryl, pyrazolyl, indolyl, isoindolyl, benzimidazolyl, purinyl, carbazolyl, 1,2,5-thiadiazolyl, quinazolinyl, pyridazinyl, pyrazinyl, cinnolinyl, phthalazinyl, quinoxalinyl, xanthinyl, hypoxanthinyl, pteridinyl, 5-azacytidinyl, 5-azauracilyl, triazolopyridinyl, imidazolopyridinyl, pyrrolopyrimidinyl, pyrazolopyrimidinyl, oxazolyl, oxadiazoyl, isoxazoyl, thiazolyl, isothiazolyl, furanyl, pyrazolyl, pyrrolyl, tetrazolyl, triazolyl, thienyl, imidazolyl, pyridinyl, and pyrimidinyl; and wherein said heterocyclic and the heterocyclic moiety of said heterocyclic-(C₁-C₈)alkyl- are selected from saturated or unsaturated nonaromatic monocyclic or bicyclic ring systems, wherein said monocyclic ring systems contain from four to seven ring carbon atoms, from one to three of which may optionally be replaced with O, N or S, and wherein said bicyclic ring systems contain from seven to twelve ring carbon atoms, from one to four of which may optionally be replaced with O, N or S; and wherein any of the aryl, heteroaryl or heterocyclic moieties of R¹ may optionally be substituted with from one to three substituents, independently selected from halo, (C₁-C₆)alkyl optionally substituted with from zero to seven fluorine atoms, phenyl, benzyl, hydroxy, acetyl, amino, cyano, nitro, (C₁-C₆)alkoxy, (C₁-C₆)alkylamino and [(C₁-C₆)alkyl]₂amino, and wherein any of alkyl moieties in R¹ may optionally be substituted with from zero to seven fluorine atoms;
R² is hydrogen, aryl, heteroaryl, heterocyclic, —SO₂R⁴, COR⁴, —CONR⁵R⁶, —COOR⁴, or —C(OH)R⁵R⁶ wherein each of R⁴, R⁵ and R⁶ is independently defined as R¹ is defined above, or R⁵ and R⁶, together with the carbon or nitrogen to which they are both attached, form a three to seven membered saturated ring containing from zero to three heterocarbons independently selected from O, N and S, and wherein said aryl, heteroaryl, and heterocyclic are defined as such terms are defined above in the definition of R¹, and wherein any of the aryl, heteroaryl and heterocyclic moieties of R² may optionally be substituted with from one to three substitutuents independently selected from halo, (C₁-C₆)alkyl optionally substituted with from zero to seven (preferably with from zero to four) fluorine atoms, phenyl, benzyl, hydroxy, acetyl, amino, cyano, nitro, (C₁-C₆)alkoxy optionally substituted with from zero to seven fluorine atoms, (C₁-C₆)alkylamino and [(C₁-C₆)alkyl]₂amino;
R³ is hydroxy, —NHSO₂R⁷, —C(OH)R⁷R⁸, fluorine or —CONHR⁷, wherein R⁷ and R⁸ are the same or different and are selected from hydrogen, (C₁-C₄)alkyl, (C₁-C₄)alkoxy and (C₁-C₄)alkoxy-(C₁-C₄)alkyl having a total of 4 or less carbon atoms, and wherein any of the alkyl moieties of R⁷ and R⁸ may optionally be substituted with from zero to seven fluorine atoms; and a pharmaceutically acceptable salt of such compound with the proviso that there are no two adjacent ring oxygen atoms and no ring oxygen atom adjacent to either a ring nitrogen atom or a ring sulfur atom in any of the heterocyclic or heteroaryl moieties of formula I.

2. A compound according to claim 1 wherein R¹ is selected from the group consisting of cyclopropylmethyl, allyl, methyl, ethyl, isopropyl, phenylethyl, and 4-pyridyl methyl;
wherein R² is selected from the group consisting of N,N-diethyl amide, N,N-methylethyl amide, diethyl carbinol, dimethyl carbinol, 2-pyridine, 3-pyridine, 2-pyrimidine, and 2-thiazole; and,
wherein R³ is selected from the group consisting of methoxy, fluorine, amide, N-methyl amide, hydroxy, methylsulfonamide, and diethylsulfonamide.

3. A pharmaceutical composition for treating a disorder or condition selected from inflammatory diseases such as arthritis, psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions, chronic pain, nonsomatic pain, acute pain and neurogenic pain, systemic lupus erythematosis, Hodgkin's disease, Sjogren's disease, epilepsy and rejection in organ transplants and skin grafts in a mammal, comprising an amount of a compound according to claim 1 that is effective in treating such disorder or condition and a pharmaceutically acceptable carrier.

4. A pharmaceutical composition for treating a disorder or condition, the treatment or prevention of which can be effected or facilitated by modulating binding to opioid receptors in a mammal, comprising an amount of a compound according to claim 1 that is effective in treating such disorder or condition and a pharmaceutically acceptable carrier.

5. A method for treating a disorder or condition selected from inflammatory diseases such as arthritis, psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions including addictions to or dependencies on alcohol, opiates, benzodiazepines, nicotine, heroin or cocaine), chronic pain, nonsomatic pain, acute pain and neurogenic pain, systemic lupus erythematosis, Hodgkin's disease, Sjogren's disease, epilepsy and rejection in organ transplants and skin grafts in a mammal, comprising administering to a mammal requiring such treatment an amount of a compound according to claim 1 that is effective in treating such disorder or condition.

6. A method for treating a disorder or condition, the treatment of which can be effected or facilitated by modulating binding to opioid receptors in a mammal, comprising administering to a mammal requiring such treatment an amount of a compound according to claim 1 that is effective in treating such disorder or condition.

7. A pharmaceutical composition for treating a disorder or condition selected from inflammatory diseases such as arthritis, psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions including addictions to or dependencies on alcohol, opiates, benzodiazepines, nicotine, heroin or cocaine, chronic pain, nonsomatic pain, acute pain and neurogenic pain, systemic lupus erythematosis, Hodgkin's disease, Sjogren's disease, epilepsy and rejection in organ transplants and skin grafts in a mammal, comprising an opioid receptor binding modulating effective amount of a compound according to claim 1 and a pharmaceutically acceptable carrier.

8. A pharmaceutical composition for treating a disorder or condition, the treatment or prevention of which can be effected or facilitated by modulating binding to opioid receptors in a mammal, comprising an opioid receptor binding modulating effective amount of a compound according to claim 1 and a pharmaceutically acceptable carrier.

9. A method for treating a disorder or condition selected from inflammatory diseases such as arthritis, psoriasis, asthma, or inflammatory bowel disease, disorders of respiratory function such as asthma, cough and apnea, allergies, gastrointestinal disorders such as gastritis, functional bowel disease, irritable bowel syndrome, functional diarrhoea, functional distension, functional pain, nonulcerogenic dyspepsia and other disorders of motility or secretion, and emesis, stroke, shock, brain edema, head trauma, spinal cord trauma, cerebral ischemia, cerebral deficits subsequent to cardiac bypass surgery and grafting, urogential tract disorders such as urinary incontinence, chemical dependencies and addictions including addictions to or dependencies on alcohol, opiates, benzodiazepines, nicotine, heroin or cocaine, chronic pain, nonsomatic pain, acute pain and neurogenic pain, systemic lupus erythematosis, Hodgkin's disease, Sjogren's disease, epilepsy and rejection in organ transplants and skin grafts in a mammal, comprising administering to a mammal requiring such treatment an opioid receptor binding modulating effective amount of a compound according to claim 1.

10. A method for treating a disorder or condition, the treatment or prevention of which can be effected or facilitated by modulating binding to opioid receptors in a mammal, comprising administering to a mammal requiring such treatment an opioid receptor binding modulating effective amount of a compound according to claim 1.
